(19) United States
(12) Patent Application Publication
(10) Pub. No.: US 2024/0366956 A1
(43) Pub. Date: Nov. 7, 2024

(54) NEAR-INFRARED SPOT LIGHT SOURCE AND CANCER TREATMENT SYSTEM

(71) Applicant: Kiwamu TAKEHISA, Kawasaki-shi, Kanagawa (JP)

(72) Inventor: Kiwamu TAKEHISA, Kawasaki-shi, Kanagawa (JP)

(21) Appl. No.: 18/586,273

(22) Filed: Feb. 23, 2024

(30) Foreign Application Priority Data
May 1, 2023 (JP) 2023-075743
Jul. 3, 2023 (JP) 2023-109213

(51) Int. Cl. A61N 5/06; B05B 17/06; G02B 17/08

(52) U.S. Cl. CPC A61N 5/0613; B05B 17/06; A61N 2005/0659; A61N 2005/0664; G02B 17/0868

(57) ABSTRACT

The disclosure relates to a cancer treatment system including a nozzle which generates the mist of a solution which consists of basic hydrogen peroxide and an imaging optics that relays an aerial image near the exit of the nozzle. The imaging optics can relay a spatial image at the outlet of the nozzle. Consequently, the 1.27-micrometer wavelength radiation is focused at where the affected part of a cancer patient is positioned.

### INCORPORATION BY REFERENCE

This application is based upon and claims the benefit of priority from Japanese patent applications No. 2023-075743, filed on May 1, 2023, and No. 2023-109213, filed on Jul. 3, 2023, the disclosure of which is incorporated herein in its entirety by reference.

### BACKGROUND

The present disclosure relates to a near-infrared spot light source and a cancer treatment system that can focus near-infrared light to a small spot.

Among various light sources capable of generating near-infrared light with a wavelength of about 1.3 m, a chemically excited iodine laser (It is commonly called COIL. COIL is an acronym for Chemical Oxygen-Iodine Laser) capable of high-power operation with a wavelength of 1.315 um is widely known. For COIL, a mixed solution of hydrogen peroxide (H₂O₂) and potassium hydroxide (KOH) or sodium hydroxide (NaOH) is used. COIL transfers the energy of an excited singlet oxygen molecule (O₂ (1Δg), (hereinafter referred to as singlet oxygen) generated by the chemical reaction between the mixed solution and chlorine gas to the iodine atom. In this way, the excited iodine atom is generated and lasing occurs. Non Patent Literature 1 (Stephen C. Hurlock, et al., “COIL technology development at Boeing,” Proceedings of SPIE Vol. 4631, 101-115 (2002)), Non Patent Literature 2 (Masamori Endo, et al., “History of COIL development in Japan: 1982-2002,” Proceedings of SPIE Vol. 4631, 116-127 (2002)), Non Patent Literature 3 (Edward A. Duff and Keith A. Truesdell, “Chemical Oxygen Iodine Laser (COIL) Technology and Development,” Proceedings of SPIE Vol. 5414, 52-68 (2004)) and Non Patent Literature 4 (Jarmila Kodymová, “COIL—Chemical Oxygen Iodine Laser: Advances in development and applications,” Proceedings of SPIE Vol. 5958, 595818 (2005)) describe chemically excited iodine lasers.

Meanwhile, a laser light source for directly lasing from singlet oxygen without transferring energy to iodine has been studied. This laser light source is called an oxygen molecule laser. However, as the oxygen molecule laser has a low gain, it is difficult for the oxygen molecule laser to generate the laser. Patent Literature 1 (International Patent Publication No. WO. 2015/114682) and Non Patent Literature 5 (“Investigation of a laser oscillator development based on singlet excited oxygen”, The Faculty of Science and Engineering, Keio University, 1986) explain the oxygen molecule laser.

The wavelength of the oxygen molecule laser is 1.27 um. The laser light with wavelength of 1.27 um is also obtained by a Raman laser based on Raman conversion and so on. The treatment of cancer by using the oxygen molecular laser with emission spectra of singlet oxygen is being investigated as an application expected to be effective. Patent Literature 2 (Japanese Patent No. 6859556) explains this application.

In the above-mentioned cancer treatment system, Patent Literature 1 discloses a cancer treatment system using an oxygen laser which produces a laser beam having a wavelength of approximately 1.27 micrometers. Since the laser has exactly the same spectrum as that of the emission from singlet oxygen, it can efficiently an excite oxygen molecule to become a single oxygen.

However, there is one problem with the above-mentioned cancer treatment apparatus using an oxygen laser. Namely, the laser gain of an oxygen laser is quite low, the laser needs to be more than several meters long, and thus it lacks portability.

### SUMMARY

The oxygen molecule laser has a low gain. Therefore, it is necessary to increase a gain length of the laser to several meters or longer in order to generate laser oscillation. However, there is a problem in this case that the cancer treatment system becomes long and large.

The purpose of this disclosure is to provide a near-infrared spot light source and a cancer treatment apparatus that can converge near-infrared light to a small spot with a small configuration. As a result, 1.27-micormeter wavelength radiation spot can be converged at the outside of the cancer treatment system by the imaging optics.

In order to achieve the above object, the near-infrared spot light source has a nozzle for generating mist of BHP solution and supplies chlorine gas in the vicinity of the outlet of the nozzle. Thus, it is possible to generate a singlet oxygen near the outlet of the nozzle. By relaying an image of the light with a wavelength of 1.27 um emitted from the singlet oxygen by using an imaging optics, it is possible to form a small spot image of the light outside the apparatus. Note that, the nozzle is a mechanical component called a spray nozzle. The nozzle has a function of producing mist from liquid and is used in various spray cans. Non Patent Literature 6 (“Development of a Mist Singlet Oxygen Generator” Japanese Journal of Applied Physics, Vol. 41, pp. 5193-5197, 2002”) disclose that the singlet oxygen is generated by the nozzle and is used for a singlet oxygen generator as a component of the COIL.

The near-infrared spot light source according to the present embodiment is different from an oxygen molecular laser in that the apparatus does not become long and large, and a small apparatus can be realized. Since the generated near-infrared light is not laser light, it is difficult to converge the light to a diameter of 1 mm or less and transmit it over a long distance by an optical fiber, for example. However, it is effective for applications such as cancer treatment where the light can be converged to a spot with a diameter of several millimeters.

According to the present disclosure, it is possible to provide a near-infrared spot light source and a cancer treatment apparatus that can converge near-infrared light to a small spot.

The above and other objects, features and advantages of the present disclosure will become more fully understood from the detailed description given herein below and the accompanying drawings which are given by way of illustration only, and thus are not to be considered as limiting the present disclosure.

### BRIEF DESCRIPTION OF THE DRAWINGS

FIG. 1 is a schematic diagram of a near-infrared spot light source according to Embodiment 1;

FIG. 2 is a schematic diagram of imaging optics of a near-infrared spot light source;

FIG. 3 is a schematic diagram of imaging optics of a near-infrared spot light source;

FIG. 4 is a schematic diagram for explaining an example of a configuration of a nozzle and its surroundings;

FIG. 5 is a schematic diagram for explaining an example of a configuration of a nozzle and its surroundings;

FIG. 6 is a schematic diagram for explaining an example of a configuration of a nozzle and its surroundings;

FIG. 7 is a schematic diagram for explaining an example of a configuration of a nozzle and its surroundings;

FIG. 8 is a cross-sectional view of a cancer treatment system 300 according to a first Embodiment;

FIG. 9 is a cross-sectional view of a cancer treatment system 300 according to a second Embodiment; and

FIG. 10 is a cross-sectional view of a cancer treatment system 300 according to a third Embodiment.

### DESCRIPTION OF EMBODIMENTS

Exemplary embodiments of the present disclosure are explained with reference to the attached drawings. The exemplary embodiments explained below are merely examples of the present disclosure, and the present disclosure is not limited to these exemplary embodiments. Note that components denoted by the same reference numerals in the specification and drawings indicate the same components.

### First Embodiment

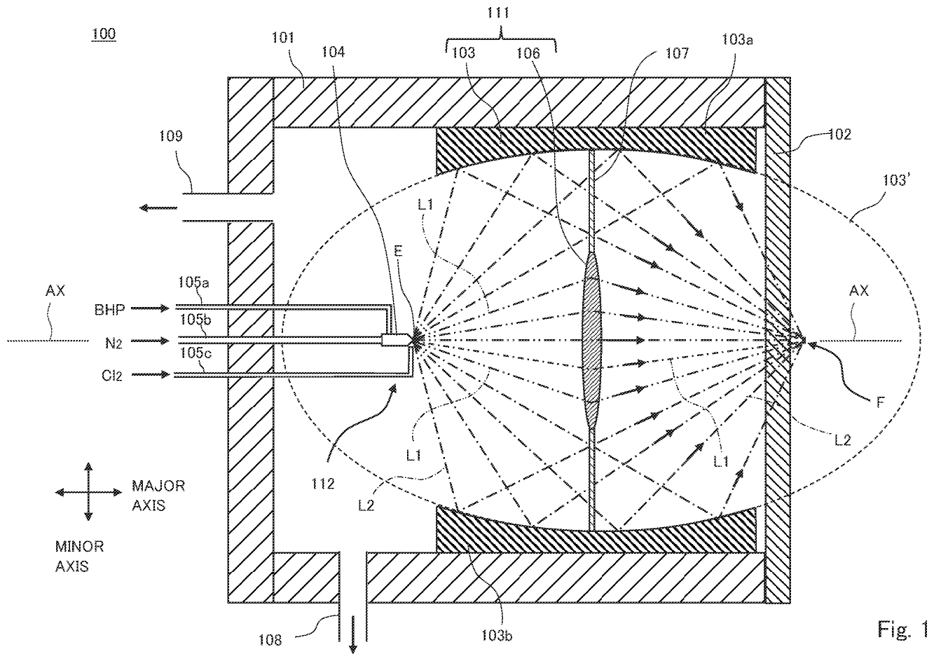

Hereinafter, the first embodiment according to the present disclosure is described based on FIG. 1. FIG. 1 is a schematic diagram which shows a cross-section of a near-infrared spot light source 100. The near-infrared spot light source 100 includes a housing 101, a window 102, an imaging optics 111 and a singlet oxygen generator 112. Here, the near-infrared spot light source 100 described below is used in a cancer treatment system. That is, the near-infrared light generated by the near-infrared spot light source 100 is irradiated to cancer cells.

In the near-infrared spot light source 100, the window 102 is attached to the cylindrical housing 101 so as to be in close contact therewith. The housing 101 and the window 102 are airtight containers. A sealing member such as an O-ring may be interposed between the housing 101 and the window 102. The window 102 is formed of a transparent resin or glass. The window 102 is formed in a parallel plate shape.

The imaging optics 111 is installed in the housing 101. The imaging optics 111 relays a spatial image formed near the outlet of the nozzle 104. The imaging optics 111 includes a spheroidal mirror 103 and a condenser lens 106. The condenser lens 106 is disposed inside the spheroidal mirror 103.

The imaging optics 111 is placed on the inside wall surface of the spheroidal mirror 103. In a cross-section parallel to a paper plane of FIG. 1, the spheroidal mirror 103 is elliptical. The spheroidal mirror 103 can relay a spatial image near the nozzle 104. In FIG. 1, a whole ellipse 103′ of the spheroidal mirror 103 is illustrated as a dash line. As illustrated by the dash line, a part of the ellipse extends outside of the housing 101. The direction of the minor axis of the ellipse is a horizontal direction, the direction of the major axis of the ellipse is a vertical direction. The spheroidal mirror 103 is formed by rotating the part of the whole ellipse 103′ around the central axis AX (a straight line through the focus spot F extending in the major axis included in the paper surface). The central axis AX is a straight line contained in the vertical direction.

The nozzle 104 is placed on the central axis AX of the ellipse. The spheroidal mirror 103 is composed of two components 103a and 103b. The component 103a is a left part of the spheroidal mirror 103. The component 103b is a right part of the spheroidal mirror 103. The two components 103a and 103b are combined and integrated to form the spheroidal mirror 103. The spheroidal mirror 103 consists of the two components 103a and 103b so as to improve the manufacturability thereof.

The singlet oxygen generator 112 is provided in the housing 101. The singlet oxygen generator 112 includes a nozzle 104, a tube 105a, a tube 105b and a tube 105c. The nozzle 104 is a two-fluid nozzle (air atomizing nozzle). Therefore, two tubes 105a and 105b are connected to the nozzle 104.

Liquid and pressured gas are supplied to the two-fluid nozzle to generate a fine particle mist. The BHP solution is supplied from the tube 105a to the nozzle 104, and nitrogen gas is supplied from the tube 105b to the nozzle 104. As a result of supplying both BHP solution and nitrogen gas, the mist of the BHP solution is ejected from the tip of the nozzle 104. Instead of the nitrogen gas, a stable gas such as helium gas can be used.

The outlet of the tube 105c is positioned in the vicinity of the outlet of the nozzle 104. That is, the tip of the tube 105c is arranged near the outlet of the nozzle 104. The chlorine gas is supplied to the tube 105c and ejected from the outlet of the tube 105c toward the misty BHP solution. The misty BHP solution reacts with the chlorine gas immediately after it is ejected from the outlet of the nozzle 104. Consequently, a singlet oxygen is generated and 1.27-micormeter wavelength near-infrared lights L1 and L2 are emitted from the singlet oxygen.

A radiation point E is at a position near the outlet of the nozzle 104 where the chlorine gas is ejected from the tube 105c. The singlet oxygen emits light with a wavelength of 1.27 um in all directions. As illustrated in FIG. 1, the lights L1 and L2 propagate and spread in an upper hemisphere with respect to the nozzle 104. L1 represents a part of the radiation while L2 represents another part of the radiation, the angle of L1 with respect to the central axis AX being smaller than the angle of L2 with respect to the central axis AX. Among the lights spreading radially, the light L1 having a small angle with respect to the central axis AX enters the condenser lens 106. The condenser lens 106 refracts the light L1. Therefore, the light L1 from the condenser lens 106 travels while converging. As a result, the light L1 is converged to the focus spot F after passing through the window 102. The focus spot F is located at a place where the treatment area is arranged. The condenser lens 106 concentrates the light L1 around the treatment area. A spatial image of the radiation point E by the imaging optics 111 is formed in the focus spot F.

The condenser lens 106 is held by a lens holder (not shown). A plurality of beams 107 is attached around the lens holder. The condenser lens 106 is fixed in the housing 101 by the beams 107. The beams 107 are, for example, rod-shaped members extending from a reflection surface of the spheroidal mirror 103 toward the periphery of the condenser lens 106. The beams 107 function as a frame for supporting the condenser lens 106. As shown in FIG. 1, the beams 107 extend from two places in the spheroidal mirror 103. However, the beams 107 may extend from three or more places in the spheroidal mirror 103. That is, three or more beams 107 may extend from the spheroidal mirror 103.

On the other hand, among the lights spreading radially, the light having a large angle with respect to the central axis AX is the light L2. The light L2 passes through the outside of the condenser lens 106. The light L2 does not enter the condenser lens but instead enters the reflecting surface the spheroidal mirror 103. The light L2 reflected by the spheroidal mirror 103 travels while converging. The whole 103 ‘of the ellipse of the spheroidal mirror 103 has two focal points. The light rays emitted from one focal point are reflected on the elliptical surface of the spheroidal mirror 103, and thus reach the other focal point. The radiation point E at the outlet of the nozzle 104 is located near one focal point, and the focus spot F is located near the other focal point. The light L2 having a wavelength of 1.27 um reflected by the spheroidal mirror 103 is focused and irradiated on the cancer cell. In this way, the imaging optics 111 can focus light L1 and L2 on the focus spot F having a diameter of several millimeters.

The near-infrared spot light source 100 can be used as a cancer treatment system. The focus spot F is the position where the treatment is provided. The cancer cell should be placed at the focus spot F by adjusting the posture of a patient. The focus spot F is located outside of the housing 101. That is, after the lights L1 and L2 pass through the window 102, the light L1 and L2 are concentrated in the focus spot F. When the posture of the patient is adjusted so that the cancer cells placed at the focus spot F, the cancer cells are irradiated with the lights L1 and L2 having a wavelength of 1.27 um emitted from the near-infrared spot light source 100. Thus, the cancer cells can be destroyed. Since the lights L1 and L2 form a small spot in the focus spot F, the damage to cells other than the cancer cells can be reduced.

An exhaust duct 109 and a drain 108 are connected to the housing 101. For example, the drain 108 is connected to the side surface of the housing 101. The drain 108 is connected to a vacuum pump or the like. Therefore, the gas in the housing 101 is discharged through the drain 108. In this way, the inside of the housing 101 is evacuated from the drain 108.

The exhaust duct 109 is connected to the bottom surface of the housing 101. Water as a byproduct of the reaction between the BHP solution and chlorine gas is discharged from the exhaust duct 109. Unreacted BHP solution is discharged from the exhaust duct 109. The discharged BHP solution may be recovered and reused.

In the near-infrared spot light source 100, the nozzle 104 is used for generating singlet oxygen. Non Patent Literature 4 discloses a singlet oxygen generator for an iodine laser having a nozzle. The configuration of the near-infrared spot light source 100 according to the present embodiment differs from that of Non Patent Literature 4 in that in the former chlorine gas is supplied from the vicinity of the outlet of the nozzle 104.

The reason for this will be explained below. When the nozzle is used for the iodine laser in Non Patent Literature 4, if the singlet oxygen is generated at a high concentration, the presence ratio of singlet oxygen may be greatly reduced in a short time. Specifically, if the singlet oxygen is generated at a high concentration when the excited iodine atom is generated by reacting with the iodine atom after the generation of singlet oxygen, the collision deactivation of the singlet oxygen group will be promoted. As a result, presence ratio of singlet oxygen may be greatly reduced before the singlet oxygen sufficiently reacts with the iodine atom. Therefore, for the iodine laser, it is necessary to supply chlorine gas after the mist of the BHP solution diffuses greatly from the nozzle.

On the other hand, in the near-infrared spot light source shown in FIG. 1, it is necessary to generate a high concentration of singlet oxygen in order to reduce a size of the region of emissions from the singlet oxygen. Therefore, the tube 105c that supplies chlorine gas is located near the outlet of the nozzle 104. The tube 105c ejects chlorine gas near the outlet of the nozzle 104. Therefore, it is possible to react the chlorine gas with the BHP solution before the mist of the BHP solution spreads widely, that is, immediately after the BHP solution is emitted from the nozzle 104.

### (Imaging Optics 111)

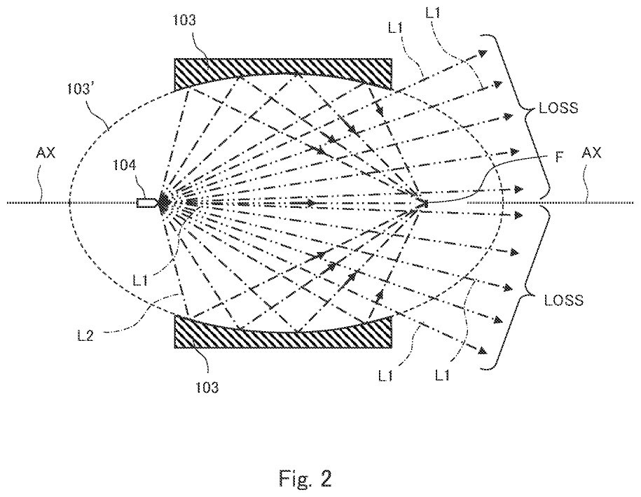

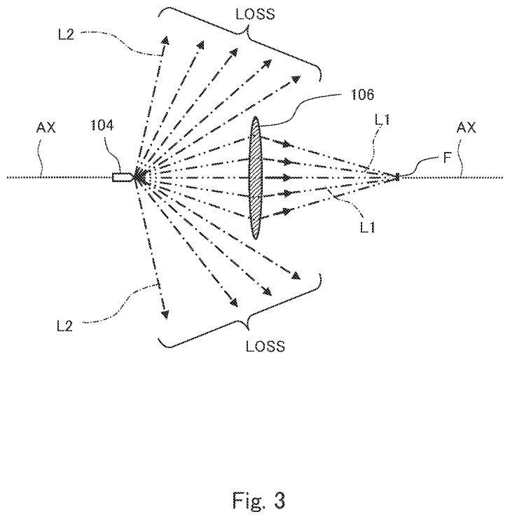

The near-infrared spot light source 100 shown in FIG. 1 includes two optical components such as the condensing lens 106 and the spheroidal mirror 103. The radiation point E is located near the outlet of the nozzle 104. The condenser lens 106 guides the light L1 having a wavelength of 1.27 um generated at the radiation point E to the focus spot F. The spheroidal mirror 103 also guides the light L1 having a wavelength of 1.27 um generated at the radiation point E to the focus spot F. The reason for using both the condenser lens 106 and the spheroidal mirror 103 for this guiding of the light will be described below with reference to FIGS. 2 and 3.

FIG. 2 is a schematic diagram showing a configuration having only the spheroidal mirror 103. FIG. 3 is a schematic diagram showing a configuration having only the condenser lens 106. When only the spheroidal mirror 103 is used, as shown in FIG. 2, the light L1 having a small spreading angle with respect to the central axis AX passes outside the focus spot F. Since the light L1 is not concentrated in the focus spot F, the light L1 is lost. Therefore, the utilization efficiency of light becomes low.

When only the condenser lens 106 is used, as shown in FIG. 3, the light L2 having a large spreading angle with respect to the central axis AX passes outside the focus spot F. Since the light L2 is not concentrated in the focus spot F, the light L2 is lost. Therefore, the utilization efficiency of light becomes low.

As described above, when only either one of the spheroidal mirror 103 or the condenser lens 106 is used, the proportion of light that is not condensed in the focus spot F increases. As a result, a large proportion of light with a wavelength of 1.27 um generated from the vicinity of the nozzle is not collected at the place where the treatment section is arranged, resulting in loss. Therefore, in the near-infrared spot light source 100 according to the present embodiment, two imaging optics 111 are formed by using two optical components such as the condensing lens 106 and the spheroidal mirror 103. The imaging optics 111 relays the spatial image of the radiation point E to the focus spot F. The focus spot F of the condensing lens 106 coincides with the focus spot F of the spheroidal mirror 103. The cancer cells can be effectively irradiated with the light L1 and the light L2.

The near-infrared spot light source 100 that generates the light with a wavelength of 1.27 um is mounted in a cancer treatment system. Since Lights L1 and L2 having a wavelength of 1.27 um are generated from singlet oxygen, the radiation consisting of many narrow lines around 1.27-micrometer in wavelength is strongly absorbed by oxygen molecules because the radiation spectrum completely matches the absorption spectrum. The lights L1 and L2 with a wavelength of 1.27 um are generated from the singlet oxygen molecules. Therefore, by irradiating the radiation around the cancer cells, oxygen molecules dissolved in the blood near the cancer cells are excited to be singlet oxygen. The cancer cells are irradiated with the light L1 and the light L2 with a wavelength of 1.27 um. The singlet oxygen molecules emit the near-infrared light (hereinafter also referred to as 1.27 um wavelength light) with a wavelength of 1.27 um. More specifically, the singlet oxygen molecules have an emission spectrum consisting of dozens of emission lines near 1.268 um, which is consistent with the absorption spectrum of ground-state oxygen molecules.

In other words, in the singlet state of an oxygen molecule, the oxygen molecule emits a photon corresponding to the energy of its excited level, so that the ground-state oxygen molecule absorbs the photon strongly. The strong absorption means that the absorption cross section is relatively large. Therefore, by irradiating the cancer cell periphery with this 1.27 um wavelength light, dissolved oxygen existing around the cancer cell is efficiently excited to the singlet state.

The reason why the oxygen molecule has many absorption lines is that its electronic states, both in the ground state and the singlet state, are separated into many rotational levels due to the fact that the oxygen molecule is a two-atom molecule. That is, the energy of the transition slightly differs due to the difference between the rotational levels of the upper and lower levels when the transition occurs upon the absorption or emission of energy. The singlet oxygen generator 112 generates excited oxygen by a chemical reaction of a BHP solution. Therefore, the output power of light having a wavelength of 1.27 um can be easily enhanced. Since 1.27 um light having a large output power can be obtained, the cancer can be treated in a short time.

### (Examples of Configuration of Singlet Oxygen Generator 112)

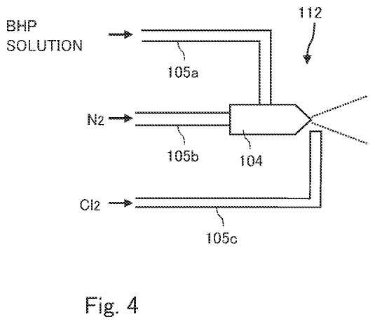

Next, examples of a configuration of singlet oxygen generator 112 will be described with reference to FIGS. 4 to 7. Four examples of the configuration are shown in FIGS. 4 to 7. Each example of the configuration will be described below.

The nozzle 104 shown in FIG. 4 has a configuration similar to that shown in FIG. 1. The nozzle 104 is a two fluid nozzle. A tube 105a and a tube 105b are connected at the nozzle 104. The tube 105a is a pipe to which the BHP solution is supplied. The tube 105b is a pipe to which the nitrogen gas is supplied.

The BHP solution is ejected from the nozzle 104 together with N₂ gas. By supplying nitrogen gas at high pressure, the nozzle 104 can make the BHP solution into a mist and eject it. In addition, the tip of the tube 105c is arranged near the ejection port of the nozzle 104. Chlorine gas is supplied to the tube 105c which is a pipe. Then, the tube 105c the ejects chlorine gas toward a mist of BHP solution ejected from the nozzle 104. Thus, the singlet oxygen can be efficiently generated.

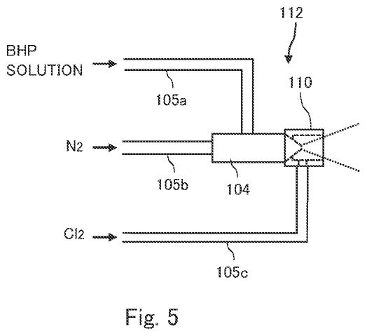

As shown in FIG. 5, a frame is added to the structure shown in FIG. 4. More particularly, a cylindrical frame 110 is attached to the tip of the tube 105c. The frame 110 is arranged in the vicinity of the ejection port of the nozzle 104. Specifically, the frame 110 is arranged to surround the ejection port of the nozzle 104. The both bottom surfaces of the frame 1110 are open. The tube 105c ejects chlorine gas from the side surface of the frame 110 into the inside of the frame 110. As a result, the chlorine gas discharged from the tube 105c efficiently contacts the mist of the BHP solution discharged from the nozzle 104. In this way, the reaction efficiency of singlet oxygen can be enhanced.

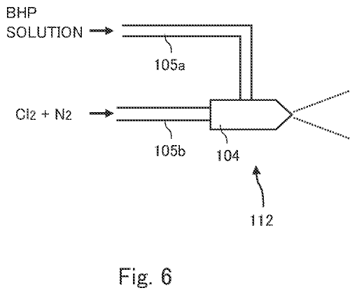

The tube 105c shown in FIG. 5 is omitted from FIG. 6. The chlorine gas is supplied to the two-fluid nozzle 104 from the tube 105b. Specifically, a mixed gas of nitrogen gas and chlorine gas is supplied to the two-fluid nozzle 104 from the tube 105b. Therefore, a high-pressure mixed gas is ejected from the nozzle 104 together with the BHP solution.

As shown in FIG. 6, the mixed gas of nitrogen gas and chlorine gas is supplied to the tube 105b, but instead only chlorine gas may be supplied to the tube 105b. Thus, the supply of nitrogen gas can be omitted. When the flow rate of chlorine gas is too high, a large amount of unreacted chlorine is generated. In this case, a gas mixed with chlorine gas and nitrogen gas may be injected into the nozzle.

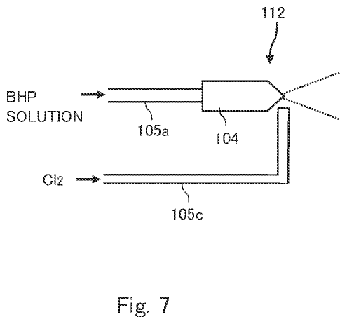

As shown in FIG. 7, a single-fluid nozzle (typical simple nozzle) is used for the nozzle 104. Only BHP solution can be supplied to the nozzle 104, which enables the nozzle 104 to be simplified. In this case, the chlorine gas is ejected from the tube 105c as shown in FIGS. 1 and 4. The outlet of the tube 105c is arranged near the outlet of the nozzle 104. Therefore, the chlorine gas is supplied so that the chlorine gas immediately contacts the mist of the BHP solution discharged from the nozzle 104. In the case where the one-fluid nozzle 104 is used, the cylindrical frame 110 may be attached to the tip of the nozzle 104 as shown in FIG. 5.

### Second Embodiment

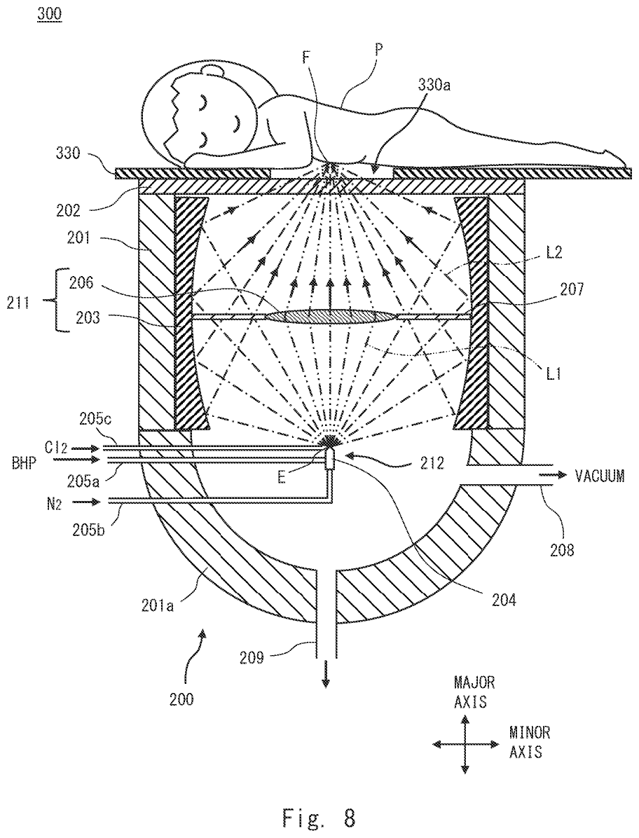

The configuration of second embodiment will be described with reference to FIG. 8. FIG. 8 is a configuration diagram showing a cross-sectional structure of a cancer treatment system 300 using a near-infrared spot light source 200. The near-infrared spot light source 200 has a different orientation from that of the near-infrared spot light source 100 shown in FIG. 1. The contents of FIG. 8 overlapping with those of FIG. 1 will be omitted. For example, the central axis AX or the like is not shown in FIG. 8.

The housing 201, the window 202, the spheroidal mirror 203, the nozzle 204, the tubes 205a to 205c, the condenser lens 206, the beam 207, the imaging optics 211, and the singlet oxygen generator 212 in the second embodiment correspond to the housing 101, the window 102, the spheroidal mirror 103, the nozzle 104, the tubes 105a to 105c, the condenser lens 106, the beam 107, the imaging optics 111, and the singlet oxygen generator 112 in the first embodiment, respectively.

In FIG. 8, the window 202 is installed on the upper surface side of the housing 201. The spheroidal mirror 203 is installed inside the side wall of the housing 201. The major axis direction of the ellipse in the cross section of the spheroidal mirror 203 is a vertical direction, and the minor axis direction is a horizontal direction. The optical axis of the condenser lens 206 is parallel to the vertical direction. The beam 207 extends in the horizontal direction.

The bottom surface 201a of the housing 201 is formed in a hemispherical shape. When the mist of the unreacted BHP solution liquefies, it falls due to gravity. The reaction product such as water also falls due to gravity. Accordingly, an exhaust duct 209 is attached to the lower end of the bottom surface 201a. Thus, the BHP solution and water can be discharged from the exhaust duct 209. The bottom surface 201a is not limited to being a hemispherical shape and may be instead an inclined flat plane or the like. The drain 208 is mounted at a position below the spheroidal mirror 203 and above the bottom surface 201a of the housing 201.

The singlet oxygen generator 212 is disposed below the imaging optics 211. The tubes 205a and 205b are connected to the nozzle 204. The nozzle 204 ejects a BHP solution upward. A tip of the tube 205c is disposed near the ejection port of the nozzle 204. The tube 205c ejects the chlorine gas from the side of the nozzle 104.

The lights L1 and L2 are focused on the focus spot F located just above the window 202 arranged on the upper part of the cancer treatment system 300. The cancer treatment system 300 has a bed 330 arranged on the upper side of the window 202. The bed 330 has an opening 330a. The lights L1 and L2 pass through the opening 330a and are irradiated to the affected area of the patient P. The opening 330a may be provided with a transparent window that transmits the lights L1 and L2.

The near-infrared spot light source 200 is suitable for the treatment of breast cancer. Since the focus spot F is located on the upper side of the cancer treatment system 300, the patient P can be treated while she is sleeping position with the affected area facing downwards. The bed 330 may also be relatively movable with respect to the near-infrared spot light source 200. For example, the irradiation position can be adjusted by changing the position of the bed 330 in the horizontal or vertical direction.

### Modified Example

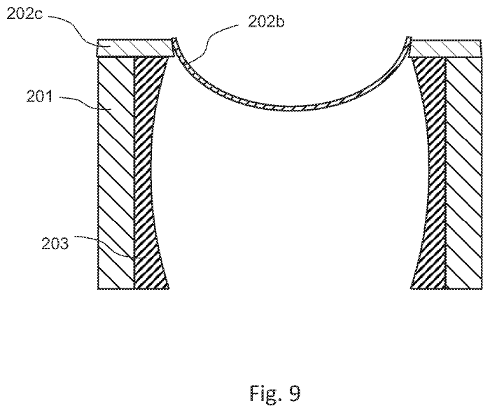

In the modified example, the shape of the window 202b is different from that of the window 202 in FIG. 8. The configuration other than the window 202b is the same as that of the second embodiment, and therefore, some components are omitted in FIG. 9.

The window 202b has a spherical shape, with one surface having a convex surface and the other surface having a concave surface. Therefore, the window 202b is a lens commonly called a meniscus lens. The peripheral part of the window 202b is held by a window holding plate 202c. The window holding plate 202c extends from the window 202b to the outer periphery of the housing 201. The window holding plate 202c is fixed to the housing 201.

In the present embodiment, the light passing through the window 202b is incident almost perpendicularly on the front surface and the rear surface of the window 202b. Therefore, the refraction of the light when it passes through the window 202b is reduced, and aberration is reduced. As a result, the expansion of the condensing spot by the aberration is suppressed. As a result, the light can be condensed to a small spot. Since the spot diameter of the light can be reduced, the cancer can be effectively treated.

An exhaust duct 209 is connected at the housing 201. Since a vacuum pump, not shown, is connected at the exhaust duct 209, the inside of the housing 201 can be evacuated. In the embodiment and a modification thereof, the near-infrared spot light source has a nozzle and an imaging optics, and supplies chlorine gas om the vicinity of the outlet of the nozzle. The nozzle ejects a mixture of an alkaline solution and hydrogen peroxide water into a mist. The imaging optics relays a spatial image near the outlet of the nozzle. Therefore, the configuration can be miniaturized and the near-infrared light can be concentrated in a minute spot.

The nozzle may be a two-fluid nozzle. The BHP solution and the chlorine gas may be supplied to the two-fluid nozzle, respectively. Thus, singlet oxygen can be efficiently generated. In addition, the imaging optics may preferably include a spheroidal mirror and condenser lens. Thus, light can be efficiently utilized.

The near-infrared spot light sources described above are suitable for cancer treatment systems such as those for treating breast cancer. The cancer treatment system has a small configuration and can irradiate cancer cells with near-infrared light in minute spots. In addition, the light with a wavelength of 1.27 um has a high transmittance in the skin. By irradiating the light of wavelength of 1.27 um wavelength light emitted from the singlet on the cancer cell, it is possible to treat the cancer efficiently. The emission line of the singlet oxygen matches the absorption spectrum of ground-state oxygen molecules. Thus, cancer can be efficiently treated. Further, therapeutic effects can be enhanced.

### Third Embodiment

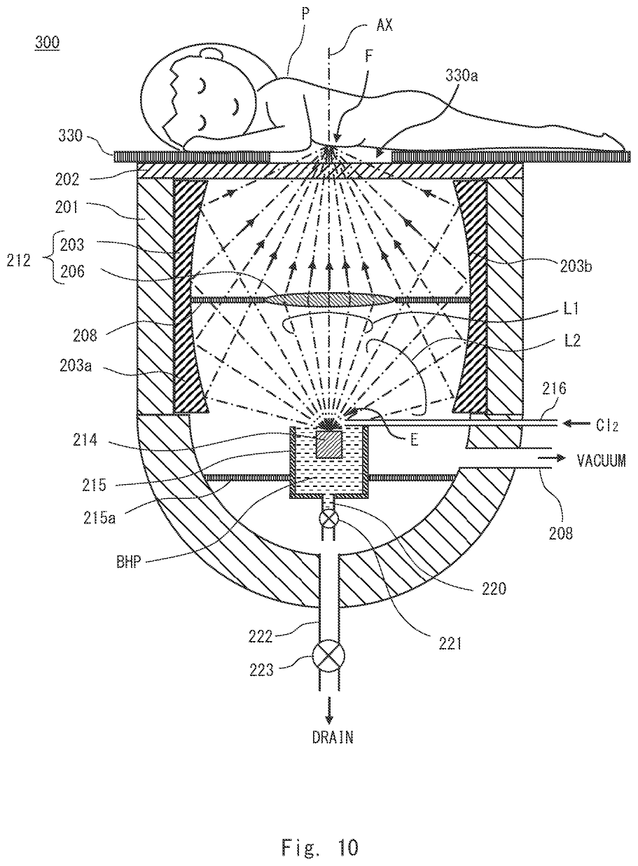

Hereinafter, the cancer treatment system 300 according to this embodiment will be described with reference to FIG. 10. FIG. 10 is a configuration diagram showing a cross-sectional structure of the cancer treatment system 300 including the near-infrared spot light source 200. The near-infrared spot light source 200 includes a housing 201, a window 202, an imaging optics 211, and an ultrasonic vibrator 214. In this embodiment, the ultrasonic vibrator 214 atomizes the BHP solution. Since the configuration other than the ultrasonic vibrator 214 is the same as that of above embodiments, the description thereof will be omitted. For example, the housing 201, the window 202, and the imaging optics 211 are the same as those of the first and second embodiments.

The ultrasonic vibrator 214 is placed on the central axis AX of an ellipse. The imaging optics 211 relays a spatial image near the outlet of the ultrasonic vibrator 214 having a diameter of 25 mm. The upper surface of the ultrasonic vibrator 213 is a vibration surface. The imaging optics relays the spatial image near the vibration surface. The ultrasonic vibrator 214 is located above the center of a cup 215 which is filled with the BHP solution. The ultrasonic vibrator 214 and the cup 215 are held in the housing 201 by the arms 215a. The vibration surface of the ultrasonic vibrator 214 is immersed in the BHP solution. As a result, the vibration of the ultrasonic vibrator 214 atomizes the BHP solution. The ultrasonic vibrator 214 generates the mist of the BHP solution. The cup 215 may be cooled from the surroundings.

On the other hand, the outlet of the tube 216 for supplying the chlorine gas is arranged on the right side of the cup 215 just above the ultrasonic vibrator 214. The tube 216 emits the chlorine gas toward the ultrasonic vibrator 214. The mist of BHP solution generated from the ultrasonic vibrator 214 reacts with the chlorine gas emitted from the outlet of the tube 216. The chemical reaction between the BHP solution and the chlorine gas produces the singlet oxygen. From the singlet oxygen, the near-infrared light with a wavelength of 1.27 um is emitted in all directions, and the light L1 and the light L2 travels upward as it spread through the upper side of the housing 201. As described in the first embodiment, the light L1 is a component emitted at a small angle with respect to the central axis AX direction, and the light L2 is a component emitted at a large angle with respect to the central axis AX direction. The radiation point E is a region where the light L1 and the light L2 are generated.

The light L1 enters the condenser lens 206. The condenser lens 206 refracts the light L1. Therefore, the light L1 from the condenser lens 206 travels while converging. As a result, the light L1 is converged to the focus spot F after passing through the window 202. The focus spot F is the place where the treatment area is arranged. The condenser lens 206 concentrates light L1 around the treatment area. A spatial image of the radiation point E formed by the imaging optics 211 is formed in the focus spot F.

On the other hand, the light L2 passes through the outside of the condenser lens 206. The light L2 does not enter the condenser lens but instead enters the reflecting surface the spheroidal mirror 203. The spheroidal mirror 203 condenses the light L2 on the focus spot F. As described above, the imaging optics 211 can focus the light L1 and L2 on the focus spot F at a size the same as that of the radiation point E. Therefore, the imaging optics can condense the light L1 and the light L2 to a size of 1 to 3 cm at the affected part of the cancer, thereby enabling the cancer to be efficiently treated.

A drain 208 is connected to the housing 201. The drain 208 is connected to a vacuum pump (not shown). Therefore, the gas in the housing 201 is discharged from the drain 208. In this way, the housing 201 is evacuated from the drain 208.

A drain pipe 220 is connected to the bottom of the cup 215. A valve 221 is attached to the drain pipe 220. A pipe 222 is connected to the bottom of the housing 201. A valve 223 is attached to the pipe 222.

During the laser operation, the valve 221 is closed. When the BHP solution is discharged after the laser operation, the valve 221 is opened so that the BHP solution accumulates at the bottom of the housing 201 and proceeds to the outside from the pipe 222. During the laser operation, the valve 223 attached to the pipe 222 is closed. After the laser operation, the valve 223 is opened so that the BHP solution can be discharged to the outside of the housing 201. On the other hand, water generated by the reaction between the BHP solution and the chlorine gas accumulates at the bottom of the housing 201. The water can be discharged from the pipe 222. The discharged BHP solution may be recovered and reused.

Some or all of the above embodiments can be combined as desirable by one of ordinary skill in the art. While the disclosure has been particularly shown and described with reference to exemplary embodiments thereof, the disclosure includes various modifications which do not negatively affect the purpose and advantages of the disclosure and is not limited to these exemplary embodiments.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A near-infrared spot light source comprising:
a mist generator which generates the mist of solution of basic hydrogen peroxide, and
an imaging optics which relays a spatial image in near an outlet of the mist generator.

2. The near-infrared spot light source according to claim 1,
wherein the mist generator includes a spray nozzle.

3. The near-infrared spot light source according to claim 2, wherein the nozzle is a two-fluid nozzle, and
the solution and gas are supplied to the two-fluid nozzle.

4. The near-infrared spot light source according to claim 3, wherein the gas supplied to the two-fluid nozzle is chlorine gas.

5. The near-infrared spot light source according to claim 1, wherein the mist generator includes an ultrasonic vibrator.

6. The near-infrared spot light source according to claim 5, further comprising a cup filled with the solution,
wherein the ultrasonic vibrator is located in the cup,
a vibration surface of the ultrasonic vibrator is immersed in the solution, and
the imaging optics relays the spatial image near the vibration surface.

7. The near-infrared spot light source according to claim 1, wherein the imaging optics includes a spheroidal mirror; and
a condenser lens located inside the spheroidal mirror;
wherein the spheroidal mirror and the condenser lens converge the light emitted from the near-infrared spot light source.

8. A cancer treatment system comprising:
a near-infrared spot light source according to claim 1,
wherein the imaging optics converges the light emitted from the near-infrared spot generator to cancer cells.

9. The cancer treatment system according to claim 8,
wherein the mist generator includes a spray nozzle,
the imaging optics relays the spatial image near an outlet of the spray nozzle.

10. The cancer treatment system according to claim 8,
wherein the mist generator includes an ultrasonic vibrator,
a vibration surface of the ultrasonic vibrator is immersed in the solution, and
the imaging optics relays the spatial image near a vibration surface of the ultrasonic vibrator.
